(19) United States
(12) Patent Application Publication
(10) Pub. No.: US 2006/0216262 A1
(43) Pub. Date: Sep. 28, 2006

(54) METHOD OF TREATING SKIN USING COMPOSITION INCLUDING CHAPERONIN PROTEINS

(76) Inventors: Hannah Sivak, Gilbert, AZ (US); Alberto Iglesias, Rosario (AR); Miguel Ballicora, Skokie, IL (US)

Correspondence Address: SNELL & WILMER;ONE ARIZONA CENTER, 400 EAST VAN BUREN, PHOENIX, AZ, 85004-2202, US

(21) Appl. No.: 11/276,056

(22) Filed: Feb. 10, 2006

Related U.S. Application Data
(60) Provisional application No. 60/652,112, filed on Feb. 10, 2005.

(51) Int. Cl. A61K 38/48; A61K 8/65

(52) U.S. Cl. 424/70.14; 424/94.64

(57) ABSTRACT

A method for the prevention and/or treatment of skin damage using chaperonin proteins in a dermatologically acceptable carrier is provided. The compositions are applied topically to treat conditions such as sunburn, aged skin, and scarred skin. The compositions may additionally include additional proteins such as rice hemoglobin, superoxide dismutase and/or catalase, as well as antioxidant molecules such as vitamin E, and vitamin C.

### CROSS REFERENCE TO RELATED APPLICATIONS

This application claims the benefit of U.S. Patent Application Ser. No. 60/652,112, entitled METHOD OF CARE AND/OR TREATMENT IF HUMAN SKIN AND SKIN OF OTHER MAMMALS USING EXTRACTS RICH IN MOLECULAR CHAPERON PROTEINS, filed Feb. 10, 2005.

### FIELD OF INVENTION

The present invention generally relates to methods of treating skin. More particularly, the invention relates to methods of treating and/or preventing damaged skin using compositions including molecular chaperonin proteins.

### BACKGROUND OF THE INVENTION

Proteins, whether they have enzymatic activity or a role in maintaining cellular or extra-cellular structure, should typically be properly folded in order to maintain optimum function. However, many proteins become misfolded during synthesis or shortly thereafter. Moreover, proteins can get misfolded even after they have been in place and performing their intended role. Some of these proteins may be intracellular and with enzymatic properties, and others may be extra-cellular and fulfilling a structural role, like collagen and elastin. Misfolding of collagen and elastin affect elasticity and volume, which in turn affects the appearance of the tissue as a whole. Scarring and skin aging are due, in part, to changes in the properties and shape of skin proteins.

Accordingly, compositions including materials to help maintain proteins in their properly folded structure are desired.

### DETAILED DESCRIPTION

The present invention provides methods of treating damaged skin and of preventing skin damage. More particularly, the invention provides methods of treating skin using compositions that include chaperonin proteins to maintain proteins in their properly folded state, which in turn promotes healthy tissue and helps prevent tissue damage.

“Molecular chaperones” or chaparonin proteins are proteins whose function is to facilitate the correct folding of other proteins, often after entering an organelle from the cytosol. Such proteins also prevent undesired protein-protein interactions and assist in refolding denatured proteins. Heat shock often affects the folding and function of many proteins, and this is why synthesis of molecular chaperonin proteins often increases after heat shock. Many other types of stress may also induce misfolding and loss of protein function.

The molecular chaperonin proteins for use with the present invention may be formed in a variety of ways. For example, the chaperonins may be formed by purification of a molecular chaperon protein from plant or animal sources or by over-expression of a molecular chaperon protein in Escherichia coli or yeast, followed by purification.

Once the protein has been purified by either method, the protein is incorporated into a coposition, such as a cream, ointment or balm, to be applied to a surface of tissue. As used in this context, the term “purified” does not only include chaperonin proteins purified to homogeneity for this application, but also includes materials including about 50% or more of the protein in the preparation. Exemplary compositions include purified chaperonins in a concentration of 0.01% to 5% (w/w), preferably about 0.1% to about 1%, and more preferably about 0.1% to about 0.5%. All percents set forth herein are in terms of weight percent of the entire composition, unless stated otherwise.

The compositions for use with the present invention also include a carrier. Suitable carriers include saline solution or other compatible liquid, creams, ointments, serums, and lotions. By way of one particular example, a cream formulation base includes: purified water, petrolatum, benzyl alcohol, stearyl alcohol, propylene glycol, isopropyl myristate, polyoxy140 stearate, carbomer 934, sodium lauryl sulfate, acetate disodium, and sodium hydroxide.

An exemplary ointment formulation base includes: white petrolatum and optionally mineral oil, and sorbitan sesquioleate.

An exemplary lotion formulation base includes carbomer 940, propylene glycol, polysorbate 40, propylene glycol stearate, cholesterol and related sterols, isopropyl myristate, sorbitan palmitate, acetyl alcohol, triethanolamine, ascorbic acid, simethicone, and purified water.

The compositions may also include additional proteins such as rice hemoglobin, superoxide dismutase and/or catalase, as well as antioxidant molecules such as vitamin E, and vitamin C.

### EXAMPLES

The following non-limiting examples illustrate exemplary compositions for use in accordance with various embodiments of the invention. These examples are merely illustrative, and it is not intended that the invention be limited to use of these examples. Compositions in accordance with the present invention may include the ingredients listed below as well as additional and/or alternative inert materials, preservatives, and other constituents typically found in compositions for treating and/or preventing similar conditions. In the cases where exemplary inert materials and/or preservatives are listed, these ingredients are merely exemplary, and it is understood that other similar ingredients may be substituted for the materials listed in the examples below.

The exemplary compositions listed below may be used for a variety of purposes. For example, the compositions can be applied to skin to facilitate healing or to prevent damage due to, for example radiation.

### Example 1

A serum product is formed by admixing the following ingredients.
1% sodium hyaluronate
0.5% purified chaperonin proteins (alpha crystalline)
3% Palmitoyl Oligopeptide Palmitoyl Tetrapeptide-3
98.5% purified water
0.5% methylparaben and propylparaben (Germaben II)

The serum of Example 1 was applied to subjects and a noticeable improvement in skin quality was observed.

### Example 2

An anti-aging cream formula (I gallon) was formed as follows.
3300 ml purified water at about 80° C.
1% chaperonins (alpha crystalline)
15 gm magnesium aluminum silicate (blend with 300 ml treated water from above)
15 gm xanthan gum
190 ml glycerin
3% Palmitoyl Oligopeptide Palmitoyl Tetrapeptide-3
combine the following cold mixture to above at low mixing speed
100 gm cetearyl alcohol (Ritachol 5000)
50 gm stearic acid
30 gm cetyl alcohol
150 ml caprylic/capric triglyceride
50 ml dioctyl ether
300 ml silicone
300 ml cyclomthicone&dimthicone copolyol
120 ml PEG 8
30 ml methylparaben and propylparaben (Germaben II)

The cream of Example 2 was applied to volunteers ranging in age from 40 to 75 years. The cream was applied to one half of each of their faces and a placebo was applied to the other half of the respective faces, for a period of two months. Appearance of wrinkles, as assessed by photography, was improved by about 45%. Assessment of skin elasticity and tone, as measured by cutometry, also showed a significant improvement for areas treated with the formula of Example 2 as compared to the placebo.

### Example 3

A body lotion was formed as follows
Heat steps 1 and 2 simultaneously.
Step 2: heat the following to 100° C. and hold at that temp.
9600 gm cetearyl stearyl polyglycoside
3200 gm glyceryl stearate
16,000 ml Caprylic/capric triglyceride
9600 ml Coco-caprylate/caprate
3200 ml Dioctyl ether
Step 1: In the steam kettle, heat 128 L. (33.8 gal) H₂O to 90° C.
First pour mixture from Step 1 into the preheated large kettle. Next, make a paste of 400 gm. XANTHAN GUM and 6.4 L of GLYCERINE. Dissolve 0.2% alpha crystalline. Dissolve 3% Palmitoyl Oligopeptide Palmitoyl Tetrapeptide-3 and add to the water.
Next, blend 400 gm sodium magnesium silicate with 16 L of the heated H₂O in a super Blender and then pour sodium magnesium silicate mixture in kettle and mix well then add mixture from Step 2 into it and mix for 20 minutes.
Next, increase the speed to 12 at the same time Add 400 ml. BENZYL ALCOHOL at 60° C. Next, mix for another 35 minutes while continuously cooling the kettle with cold tap water.
Add 800 ml methyparaben/propylparaben at 40° C.

The lotion of Example 3 was applied to subjects and a noticeable improvement in skin quality was observed.

In accordance with one embodiment of the invention, a method of preventing skin damage includes applying a composition including chaperonin proteins to an area on the skin. In accordance with one aspect of this embodiment, the method includes applying a cream to the surface of the skin. In accordance with another aspect, the method includes applying an serum to the area. In accordance with yet another aspect of this embodiment, the method includes applying a lotion of the area.

In accordance with another embodiment of the invention, a method of treating damaged skin includes applying a composition including chaperonin proteins to an area of damaged skin. In accordance with one aspect of this embodiment, the method includes applying a cream to the surface of the skin. In accordance with another aspect, the method includes applying an serum to the area. In accordance with yet another aspect of this embodiment, the method includes applying a lotion of the area.

Although exemplary embodiments of the present invention are set forth herein, it should be appreciated that the invention is not so limited. Various modifications, variations, and enhancements in the composition and method set forth herein may be made without departing from the spirit and scope of the present invention.

We claim:

1. A method of preventing skin damage, the method comprising the steps of:
providing a composition including about 0.01 wt % to about 5 wt % chaperonin proteins, and
applying the composition to an area of skin to be treated for the prevention of damage.

2. The method of claim 1, wherein the step of providing a composition comprises providing a composition comprising about 0.1 wt % to about 1 wt % chaperonin proteins.

3. The method of claim 1, wherein the step of providing a composition comprises providing a composition comprising about 0.1 wt % to about 0.5 wt % chaperonin proteins.

4. The method of claim 1, wherein the step of providing a composition comprises providing a serum.

5. The method of claim 4, wherein the step of providing a composition comprises providing a composition including about 0.5 wt % chaperonin proteins.

6. The method of claim 1, wherein the step of providing a composition comprises providing a cream.

7. The method of claim 6, wherein the step of providing a composition comprises providing a composition including about 1 wt % chaperonin proteins.

8. The method of claim 1, wherein the step of providing a composition comprises providing a lotion.

9. The method of claim 8, wherein the step of providing a composition comprises providing a composition including about 0.2 wt % chaperonin proteins.

10. A method of treating damaged, the method comprising the steps of:
providing a composition including about 0.01 wt % to about 5 wt % chaperonin proteins, and
applying the composition to an area of skin to be treated for damaged skin.

11. The method of claim 10, wherein the step of providing a composition comprises providing a composition comprising about 0.1 wt % to about 1 wt % chaperonin proteins.

12. The method of claim 10, wherein the step of providing a composition comprises providing a composition comprising about 0.1 wt % to about 0.5 wt % chaperonin proteins.

13. The method of claim 10, wherein the step of providing a composition comprises providing a serum.

14. The method of claim 13, wherein the step of providing a composition comprises providing a composition including about 0.5 wt % chaperonin proteins.

15. The method of claim 10, wherein the step of providing a composition comprises providing a cream.

16. The method of claim 15, wherein the step of providing a composition comprises providing a composition including about 1 wt % chaperonin proteins.

17. The method of claim 10, wherein the step of providing a composition comprises providing a lotion.

18. The method of claim 17, wherein the step of providing a composition comprises providing a composition including about 0.2 wt % chaperonin proteins.

19. A method of reducing an appearance of wrinkles on skin, the method comprising the steps of:
providing a composition including about 0.01 wt % to about 5 wt % chaperonin proteins, and
applying the composition to an area of skin to be treated for the reduction of appearance of wrinkles.

20. The method of claim 19, wherein the step of providing comprises providing a composition in the form selected from the group consisting of cream, serum, and lotion.
